(19) United States
(12) Patent Application Publication
(10) Pub. No.: US 2016/0287634 A1
(43) Pub. Date: Oct. 6, 2016

(54) METHOD OF MANUFACTURING AN AFOD INTRAVENOUS INJECTION FROM FRACTION IV TO PREVENT AND KILL HIV-1 AND HIV-2

(71) Applicant: Kieu Hoang, Agoura Hills, CA (US)

(72) Inventor: Kieu Hoang, Agoura Hills, CA (US)

(21) Appl. No.: 15/090,019

(22) Filed: Apr. 4, 2016

Related U.S. Application Data
(60) Provisional application No. 62/142,197, filed on Apr. 2, 2015.

(51) Int. Cl. A61K 35/16; A61K 47/42; A61K 9/08; A61K 9/00; A61K 9/19

(52) U.S. Cl. CPC A61K 35/16; A61K 9/0019; A61K 9/19; A61K 9/08; A61K 47/42

(57) ABSTRACT

The present subject matter relates to a method of manufacturing an AFOD intravenous injection, comprising dissolving a Fraction IV1+IV4 paste with WFI; adding sodium acetate, adjusting pH and agitating until fully dissolved; cooling; performing press filtration; collecting an AFOD paste comprising newly-found proteins KH 24, KH 25, KH 26, and KH 27; dissolving the paste with a buffer; centrifuging; filtrating with a depth filter; adding Tween-80; cooling; adjusting pH and adding a cold alcohol while cooling; centrifuging to obtain a second AFOD paste; dissolving the second paste with a buffer and adjusting pH; filtrating with a depth filter; ultra-filtrating; undergoing dialysis with WFI; nano filtrating for virus removal; concentrating and adjusting pH; adding a stabilizer; and filling and performing sterile filtration to obtain the AFOD intravenous injection. The present subject matter relates to an AFOD intravenous injection in liquid or lyophilized form to prevent and kill HIV-1 and HIV-2.

### RELATED APPLICATIONS

The present patent application claims priority to provisional U.S. Patent Application No. 62/142,197 filed Apr. 2, 2015, which was filed by the inventor hereof and is incorporated by reference herein in its entirety.

### TECHNICAL FIELD

The present subject matter relates to an AFOD intravenous injection obtained from Fraction IV and having newly-found proteins KH 24, KH 25, KH 26, and KH 27. In particular, the present subject matter is associated with methods of treating health conditions, particularly viruses HIV-1 and HIV-2.

### BACKGROUND

Fraction IV (Fr. IV) is a discard fraction in the plasma derived products industry. It mainly contains human albumin, apolipoprotein, transferrin, alpha 1 antitrypsin haptoglobin, vimentin, and new found proteins.

Human immunodeficiency virus (HIV) attacks the immune system, which is the body's natural defense against illness. If a person is infected with HIV, it becomes harder to fight off infections and diseases. HIV-1 and HIV-2 are two distinct strains of the virus, with HIV-1 being more predominant worldwide and HIV-2 being concentrated in western Africa.

### SUMMARY

AFOD is a novel plasma-derived product from Fraction IV of human plasma. AFOD contains 15 human proteins, of which four are newly-found proteins KH 24, KH 25, KH 26, and KH 27. According to the present subject matter, AFOD may be recovered from Fraction IV paste, which includes 15 existing and newly found proteins for intravenous injection against HIV. Of the proteins, 11 are existing proteins and four are newly-found proteins KH 24, KH 25, KH 26, and KH 27.

The final product of the present subject matter not only stops replication of HIV-1 and HIV-2, but also kills HIV-1 and HIV-2. Thus, HIV-1 and HIV-2 infections may be eradicated and prevented from the world.

An embodiment of the present subject matter is directed to a method of manufacturing an AFOD intravenous injection, comprising the steps:

a) dissolving a Fraction IV1+IV4 paste with cold water for injection (WFI) at a dilution ratio of 1:9 to create a suspension;

b) adding sodium acetate to the suspension to reach a concentration of 20 mM, adjusting the pH value of the suspension to about 3-8, and agitating until fully dissolved;

c) cooling the suspension to a temperature of 0-20° C.;

d) performing press filtration with a filter;

e) collecting a first AFOD paste comprising newly-found proteins KH 24, KH 25, KH 26, and KH 27;

f) dissolving the first AFOD paste with a TRIS-HCL buffer at pH 3-8 and a dilution ratio of 1:9;

g) centrifuging the first AFOD paste at a temperature of 10° C. to obtain a supernatant;

h) filtrating the supernatant with a depth filter to obtain a first clear filtrate;

i) adding Tween-80 to the first clear filtrate to reach a concentration of 1 wt % and TNBP to a concentration of 0.3 wt % while maintaining a solution at the temperature of 25° C. for 6 hours;

j) cooling the solution to the temperature of 1° C.;

k) adjusting pH to about 3-8 and adding a cold alcohol to a concentration of 10-40 wt % while cooling until the temperature is −5° C.;

l) centrifuging to obtain a second AFOD paste;

m) dissolving the second AFOD paste with a TRIS-HCL buffer at pH 3-8 at a dilution ratio of 1:50 and adjusting the pH to about 3-8 to obtain a second solution;

n) filtrating the second solution with a depth filter to obtain a second clear filtrate;

o) ultra-filtrating the second clear filtrate to a concentration of 3 wt % with an ultra-filtration membrane;

p) undergoing dialysis of the second clear filtrate with 10 vol % of cold WFI;

q) nano filtrating the second clear filtrate with a 20 nm filter for virus removal;

r) concentrating the second clear filtrate to 7.5 wt % protein and adjusting the pH to about 7;

s) adding albumin to the second clear filtrate to a concentration of 2.5 wt % as a stabilizer; and

t) filling and performing sterile filtration of the second clear filtrate to obtain the AFOD intravenous injection.

An embodiment of the present subject matter is directed to a method of treatment for a patient comprising administering the AFOD intravenous injection obtained from the method of manufacturing an AFOD intravenous injection to a patient in need thereof, wherein the AFOD intravenous injection transforms or repairs damaged and sick cells to become healthy cells, wherein the AFOD intravenous injection protects cellular alterations, and wherein the AFOD intravenous injection sends signals to the patient's body to produce new cells that are healthy, thereby preventing the new cells from being affected by intracellular and extracellular damaging signals.

An embodiment of the present subject matter is directed to a method of stopping replication of HIV-1 and HIV-2 in a patient comprising administering the AFOD intravenous injection obtained from the method of manufacturing an AFOD intravenous injection to a patient in need thereof.

An embodiment of the present subject matter is directed to a method of killing HIV-1 and HIV-2 in a patient comprising administering the AFOD intravenous injection obtained from the method of manufacturing an AFOD intravenous injection to a patient in need thereof.

An embodiment of the present subject matter is directed to a method of preventing infection of HIV-1 and HIV-2 in a patient comprising administering the AFOD intravenous injection obtained from the method of manufacturing an AFOD intravenous injection to a patient in need thereof.

An embodiment of the present subject matter is directed to an AFOD intravenous injection produced according to the method of manufacturing an AFOD intravenous injection.

### BRIEF DESCRIPTION OF THE DRAWINGS

FIG. 1 is a flow chart depicting the production of AFOD from Fraction IV (AFOD RAAS).

FIG. 2 shows 2D electropherosis of Fraction IV and AFOD from Fraction IV (AFOD RAAS), which shows newly-found proteins KH 24, KH 25, KH 26, and KH 27.

FIG. 3 shows the inhibition rate of AFCC RAAS in five HIV-1 strains and the control virus AMLV. Results show the inhibition rate is about 60% when the dilution is less than 1:40. Inhibition is also observed in the control virus AMLV. Cell toxicity was found in high concentrations via observation of cell morphology 48 hours after treatment.

FIG. 4 shows the results of a cell toxicity test of AFCCKH, AFOD RAAS 101, and AFCC RAAS. Test samples were diluted at 1:1/5 to start and then 1:4.5, 1:13.5, 1:40.5, 1:121.5, 1:364.5, 1:1093.5, and 1:3280.5, where the dilution was three-fold with eight dilutions in total. Cell counting kit 8 (CCK-8) was the test kit used, and the procedure was performed according to the manufacturer's manual. Results show some cell toxicity of RAAS, which likely causes the inhibition of HIV.

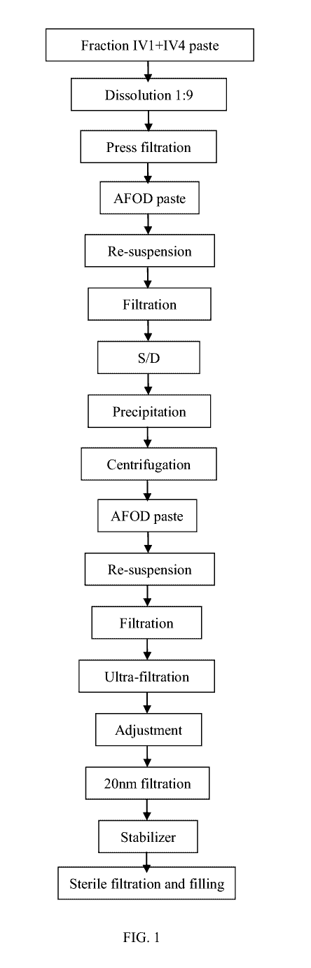

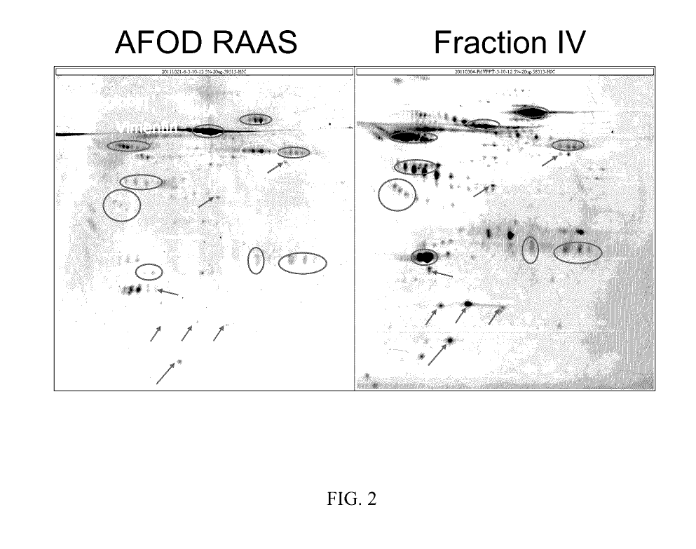

### DETAILED DESCRIPTION

Unless defined otherwise all technical and scientific terms used herein have the same meaning as commonly understood to one of ordinary skill in the art to which the presently described subject matter pertains.

Where a range of values is provided, for example, concentration ranges, percentage ranges, or ratio ranges, it is understood that each intervening value, to the tenth of the unit of the lower limit, unless the context clearly dictates otherwise, between the upper and lower limit of that range and any other stated or intervening value in that stated range, is encompassed within the described subject matter. The upper and lower limits of these smaller ranges may independently be included in the smaller ranges, and such embodiments are also encompassed within the described subject matter, subject to any specifically excluded limit in the stated range. Where the stated range includes one or both of the limits, ranges excluding either or both of those included limits are also included in the described subject matter.

Throughout the application, descriptions of various embodiments use “comprising” language; however, it will be understood by one of skill in the art, that in some specific instances, an embodiment can alternatively be described using the language “consisting essentially of” or “consisting of”.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

An embodiment of the present subject matter is directed to a method of manufacturing an AFOD intravenous injection, comprising the steps:

a) dissolving a Fraction IV1+IV4 paste with cold water for injection (WFI) at a dilution ratio of 1:9 to create a suspension;

b) adding sodium acetate to the suspension to reach a concentration of 20 mM, adjusting the pH value of the suspension to about 3-8, and agitating until fully dissolved;

c) cooling the suspension to a temperature of 0-20° C.;

d) performing press filtration with a filter;

e) collecting a first AFOD paste comprising newly-found proteins KH 24, KH 25, KH 26, and KH 27;

f) dissolving the first AFOD paste with a TRIS-HCL buffer at pH 3-8 and a dilution ratio of 1:9;

g) centrifuging the first AFOD paste at a temperature of 10° C. to obtain a supernatant;

h) filtrating the supernatant with a depth filter to obtain a first clear filtrate;

i) adding Tween-80 to the first clear filtrate to reach a concentration of 1 wt % and TNBP to a concentration of 0.3 wt % while maintaining a solution at the temperature of 25° C. for 6 hours;

j) cooling the solution to the temperature of 1° C.;

k) adjusting pH to about 3-8 and adding a cold alcohol to a concentration of 10-40 wt % while cooling until the temperature is −5° C.;

l) centrifuging to obtain a second AFOD paste;

m) dissolving the second AFOD paste with a TRIS-HCL buffer at pH 3-8 at a dilution ratio of 1:50 and adjusting the pH to about 3-8 to obtain a second solution;

n) filtrating the second solution with a depth filter to obtain a second clear filtrate;

o) ultra-filtrating the second clear filtrate to a concentration of 3 wt % with an ultra-filtration membrane;

p) undergoing dialysis of the second clear filtrate with 10 vol % of cold WFI;

q) nano filtrating the second clear filtrate with a 20 nm filter for virus removal;

r) concentrating the second clear filtrate to 7.5 wt % protein and adjusting the pH to about 7;

s) adding albumin to the second clear filtrate to a concentration of 2.5 wt % as a stabilizer; and

t) filling and performing sterile filtration of the second clear filtrate to obtain the AFOD intravenous injection.

In an embodiment, step d) is selected from the group consisting of endures, s-100, and 0.45 μm. In an embodiment, step h) further comprises filtrating with the depth filter at 10 CP+90 SP. In an embodiment, the the supernatant is then filtered at 0.45 μm. In an embodiment, step n) further comprises filtrating with the depth filter at 10 CP+90 SP. In an embodiment, the second solution is then filtered at 0.45 μm.

An embodiment of the present subject matter is directed to a method of treatment for a patient comprising administering the AFOD intravenous injection obtained from the method of manufacturing an AFOD intravenous injection to a patient in need thereof, wherein the AFOD intravenous injection transforms or repairs damaged and sick cells to become healthy cells, wherein the AFOD intravenous injection protects cellular alterations, and wherein the AFOD intravenous injection sends signals to the patient's body to produce new cells that are healthy, thereby preventing the new cells from being affected by intracellular and extracellular damaging signals.

An embodiment of the present subject matter is directed to a method of stopping replication of HIV-1 and HIV-2 in a patient comprising administering the AFOD intravenous injection obtained from the method of manufacturing an AFOD intravenous injection to a patient in need thereof.

An embodiment of the present subject matter is directed to a method of killing HIV-1 and HIV-2 in a patient comprising administering the AFOD intravenous injection obtained from the method of manufacturing an AFOD intravenous injection to a patient in need thereof.

An embodiment of the present subject matter is directed to a method of preventing infection of HIV-1 and HIV-2 in a patient comprising administering the AFOD intravenous injection obtained from the method of manufacturing an AFOD intravenous injection to a patient in need thereof.

An embodiment of the present subject matter is directed to an AFOD intravenous injection produced according to the method of manufacturing an AFOD intravenous injection. In an embodiment, the AFOD intravenous injection is in liquid form. In an embodiment, the AFOD intravenous injection is in lyophilized form.

In an embodiment, any of these or any combination of the four newly-found proteins has the ability to stop replication of HIV. In an embodiment, any of these or any combination of the four newly-found proteins has the ability to kill HIV-1 and HIV-2. In an embodiment, any of these or any combination of the four newly-found proteins has the ability to prevent infection of HIV-1 and HIV-2. In an embodiment, any of these or any combination of the newly-found proteins has the following abilities: 1) transform/repair DAMAGED and SICK cells to become KH good healthy cells, 2) protect cellular alterations, and 3) signal the body to produce new, healthy cells immunized from intracellular and extracellular damaging signals. In an embodiment, any of these or any combination of the 15 proteins in AFOD from Fr. IV has the ability to stop replication of HIV-1 and HIV-2, kill HIV-1 and HIV-2, and prevent infection of HIV-1 and HIV-2.

### EXAMPLES

In Vitro Testing

AFOD with four newly-found proteins was tested by the Comprehensive National AIDS Research Center, Tsinghua University in China, which concluded that AFOD having code name AFOD RAAS has the ability to stop replication and kill HIV.

The supplementary results of neutralization of HIV-1 Env-pseudotyped virus follow.

Samples and Control

The test samples used were AFCC RAAS.

Five virus strains were tested. The strains tested were the BC recombinant subtype virus strains CNE15 and CNE30, CRFO1 AE recombinant subtype virus CNE55, and the standard HIV-1 strains HXB2 and JRFL. All of the aforementioned HIV-1 virus strains have CCR5 receptor affinity, with the exception of HXB2, which has CXCR4 receptor affinity.

The control virus used was AMLV.

Test Method

Test samples were diluted at 1:1.5 to start. The test samples were then diluted at 1:4.5, 1:13.5, 1:40.5, 1:121.5, 1:364.5, 1:1093.5, and 1:3280.5 for a three-fold dilution, with eight dilutions in total.

Results

FIG. 3 shows the inhibition rate of AFCC RAAS in the five HIV-1 strains and the control virus AMLV. Results show the inhibition rate is about 60% when the dilution was less than 1:40, and the inhibition also was observed in the control virus AMLV. Cell toxicity was found in high concentrations via observing cell morphology 48 hours after treatment. The cell toxicity test was then conducted.

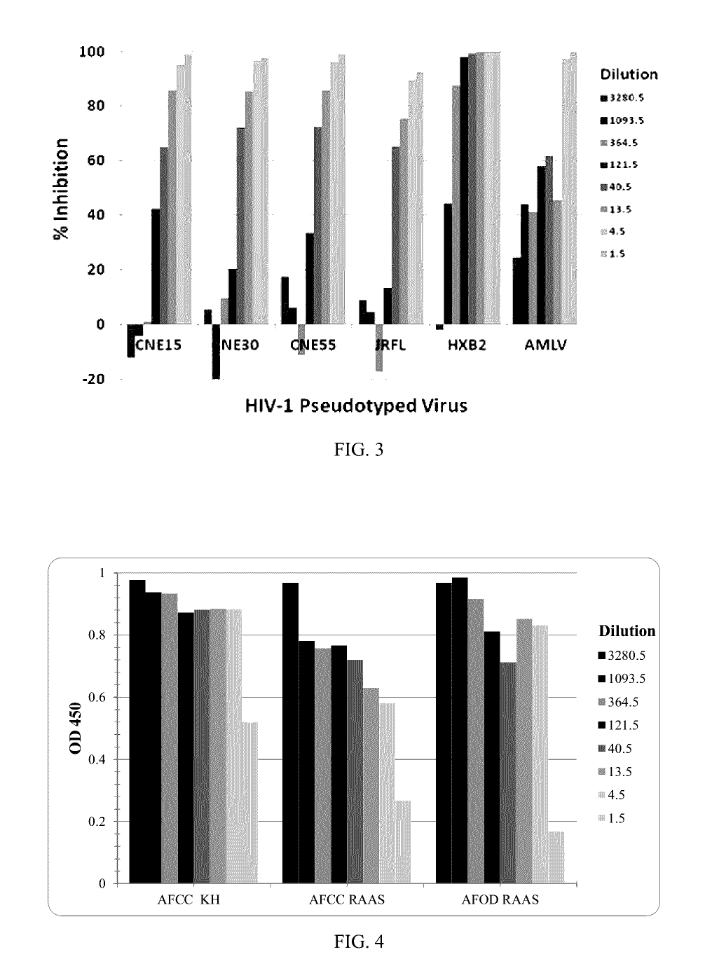

FIG. 4 shows the results of the cell toxicity test. The toxicity of AFCCKH, AFOD RAAS 101, and AFCC RAAS was tested. Test samples were diluted at 1:1.5 to start and then 1:4.5, 1:13.5, 1:40.5, 1:121.5, 1:364.5, 1:1093.5, and 1:3280.5. The dilution was three-fold with eight dilutions in total. Cell counting kit 8 (CCK-8) was the test kit used, and the procedure was carried out according to the manufacturer's manual. Results show there is some cell toxicity of RAAS. The inhibition of HIV virus likely is caused by cell toxicity.

In an embodiment, to decrease the toxicyte to cell and ensure the high inhibition of virus at high protein concentration, the protein concentration may be further increased. Further, in an embodiment, the cell culture medium (DMEM+1-% FBS) may be used as the diluent of products when preparing the samples.

According to an embodiment of the present subject matter ______.

Testimonials

An embodiment of the present subject matter is directed to a ______.

With the information contained herein, various departures from precise descriptions of the present subject matter will be readily apparent to those skilled in the art to which the present subject matter pertains, without departing from the spirit and the scope of the below claims. The present subject matter is not considered limited in scope to the procedures, properties, or components defined, since the preferred embodiments and other descriptions are intended only to be illustrative of particular aspects of the presently provided subject matter. Indeed, various modifications of the described modes for carrying out the present subject matter which are obvious to those skilled in chemistry, biochemistry, or related fields are intended to be within the scope of the following claims.

I claim:

1. A method of manufacturing an AFOD intravenous injection, comprising the steps:
a) dissolving a Fraction IV1+IV4 paste with cold water for injection (WFI) at a dilution ratio of 1:9 to create a suspension;
b) adding sodium acetate to the suspension to reach a concentration of 20 mM, adjusting the pH of the suspension to about 3-8, and agitating until fully dissolved;
c) cooling the suspension to a temperature of 0-20° C.;
d) performing press filtration with a filter;
e) collecting a first AFOD paste comprising newly-found proteins KH 24, KH 25, KH 26, and KH 27;
f) dissolving the first AFOD paste with a TRIS-HCL buffer at pH 3-8 and a dilution ratio of 1:9;
g) centrifuging the first AFOD paste at a temperature of 10° C. to obtain a supernatant;
h) filtrating the supernatant with a depth filter to obtain a first clear filtrate;
i) adding Tween-80 to the first clear filtrate to reach a concentration of 1 wt % and TNBP to a concentration of 0.3 wt % while maintaining a solution at the temperature of 25° C. for 6 hours;
j) cooling the solution to the temperature of 1° C.;
k) adjusting pH to about 3-8 and adding a cold alcohol to a concentration of 10-40 wt % while cooling until the temperature is −5° C.;
1) centrifuging to obtain a second AFOD paste;
m) dissolving the second AFOD paste with a TRIS-HCL buffer at pH 3-8 at a dilution ratio of 1:50 and adjusting the pH to about 3-8 to obtain a second solution;
n) filtrating the second solution with a depth filter to obtain a second clear filtrate;
o) ultra-filtrating the second clear filtrate to a concentration of 3 wt % with an ultra-filtration membrane;
p) undergoing dialysis of the second clear filtrate with 10 vol % of cold WFI;
q) nano filtrating the second clear filtrate with a 20 nm filter for virus removal;
r) concentrating the second clear filtrate to 7.5 wt % protein and adjusting the pH to about 7;
s) adding albumin to the second clear filtrate to a concentration of 2.5 wt % as a stabilizer; and
t) filling and performing sterile filtration of the second clear filtrate to obtain the AFOD intravenous injection.

2. The method of claim 1 wherein the filter of step d) is selected from the group consisting of endures, s-100, and 0.45 μm.

3. The method of claim 1, wherein step h) further comprises filtrating with the depth filter at 10 CP+90 SP.

4. The method of claim 3, wherein the supernatant is then filtered at 0.45 μm.

5. The method of claim 1, wherein step n) further comprises filtrating with the depth filter at 10 CP+90 SP.

6. The method of claim 5, wherein the second solution is then filtered at 0.45 μm.

7. A method of treatment for a patient comprising administering the AFOD intravenous injection obtained from the method of claim 1 to a patient in need thereof,
wherein the AFOD intravenous injection transforms or repairs damaged and sick cells to become healthy cells,
wherein the AFOD intravenous injection protects cellular alterations, and
wherein the AFOD intravenous injection sends signals to the patient's body to produce new cells that are healthy, thereby preventing the new cells from being affected by intracellular and extracellular damaging signals.

8. A method of stopping replication of HIV-1 and HIV-2 in a patient comprising administering the AFOD intravenous injection obtained from the method of claim 1 to a patient in need thereof.

9. A method of killing HIV-1 and HIV-2 in a patient comprising administering the AFOD intravenous injection obtained from the method of claim 1 to a patient in need thereof.

10. A method of preventing infection of HIV-1 and HIV-2 in a patient comprising administering the AFOD intravenous injection obtained from the method of claim 1 to a patient in need thereof.

11. An AFOD intravenous injection produced according to the method of claim 1.

12. The AFOD intravenous injection of claim 11, wherein the AFOD intravenous injection is in liquid form.

13. The AFOD intravenous injection of claim 11, wherein the AFOD intravenous injection is in lyophilized form.
